(19) United States
(12) Patent Application Publication
(10) Pub. No.: US 2024/0207276 A1
(43) Pub. Date: Jun. 27, 2024

(54) COMBINATION COMPRISING A NEUROTENSIN RECEPTOR BINDING COMPOUND AND FOLFIRINOX

(71) Applicant: Fusion Pharmaceuticals Inc., Hamilton (CA)

(72) Inventors: Diane-Charlotte Imbs, Paris (FR); Elodie Lewkowicz, Paris (FR); Alexander McEwan, Toronto (CA); Thomas Rohban, Saint Germain En Laye (FR); Sylvie Rolland, Marolles en Hurepoix (FR); Daniel Stevens, Oxfordshire (GB); John R. Forbes, Burlington (CA)

(21) Appl. No.: 18/541,584

(22) Filed: Dec. 15, 2023

Related U.S. Application Data
(63) Continuation of application No. PCT/CA2022/050961, filed on Jun. 16, 2022.
(60) Provisional application No. 63/211,312, filed on Jun. 16, 2021.

(51) Int. Cl. A61K 31/519; A61K 31/282; A61K 31/4745; A61K 31/513; A61K 51/04; A61P 35/00

(52) U.S. Cl. CPC A61K 31/519; A61K 31/282; A61K 31/4745; A61K 31/513; A61K 51/0497; A61P 35/00

(57) ABSTRACT

The present invention relates to a combination comprising a neurotensin receptor binding compound and FOLFIRINOX for use for the treatment of a neurotensin receptor overexpressing tumour in a subject.

### RELATED APPLICATION

This application is a continuation of International Application No. PCT/CA2022/050961 filed Jun. 16, 2022, which claims the benefit of and priority to U.S. Provisional Patent Application No. 63/211,312, filed on Jun. 16, 2021, the contents of each of which are hereby incorporated by reference for all purposes.

### BACKGROUND

The present invention relates to methods of treating tumours that overexpress neurotensin receptors, e.g. pancreatic cancer. In particular, the invention provides novel therapies based on the combination of a neurotensin receptor binding compound and chemotherapy, wherein said chemotherapy is the combination of folinic acid, fluorouracil, irinotecan and oxaliplatin.

Neurotensin (“NT”) is a 13 amino acid neuropeptide that is implicated in the regulation of luteinizing hormone and prolactin release and has significant interaction with the dopaminergic system.

Neurotensin binds to neurotensin receptors. Three neurotensin receptors are known, namely neurotensin receptor 1, also referred to as NTR1, neurotensin receptor 2, also referred to as NTR2, and neurotensin receptor 3, also referred to as NTR3. These neurotensin receptors are transmembrane receptors that bind the neurotransmitter neurotensin (Vincent et al., Trends Pharmacol. Sci., 1999, 20, 302-309; Pelaprat, Peptides, 2006, 27, 2476-2487). NTR1 and NTR2 which are encoded by the NTSR1 and NTSR2 genes, contain seven transmembrane helices and are G protein coupled. NTR3 has a single transmembrane domain and is encoded by the SORT1 gene.

The neurotensin receptor 1 (NTR1) is expressed predominantly in the central nervous system and intestine (smooth muscle, mucosa and nerve cells). Apart from the central nervous system, NTR1 is highly expressed in a mammalian body and a human body in particular on several neoplastic cells in several tumor indications, whereas the expression of NTR1 in most other tissues of the mammalian and the human body is either not existent or low. Under physiological conditions, weak or moderate expression of NTR1 is described only for colon.

The combination of the invention intends to treat the tumours that overexpress neurotensin receptors. By “overexpress”, it is meant a level of expression higher than in normal cells.

A “neurotensin overexpressing tumour” is also referred to as a “neurotensin positive tumour”, such as a “NTR1-positive tumour” or a “NTR1+ tumour”.

Binding and pharmacological studies demonstrate that neurotensin receptor 2 (NTR2) binds neurotensin as well as several other ligands already described for NTR1. However, unlike NTR1, NTR2 recognizes, with high affinity, levocabastine, a histamine H1 receptor antagonist previously shown to compete with neurotensin for low-affinity binding sites in the central nervous system.

The international application WO2014/086499 describes a table summarizing the expression of NTR1 as described in the prior art indicating the tissue, degree of expression, detection method and the respective references. This table is incorporated by reference.

The tumour indications where NTR1 is overexpressed include but are not limited to pancreatic ductal adenocarcinoma, small cell lung cancer, prostate cancer, colorectal cancer, breast cancer, meningioma, Ewing's sarcoma, pleural mesothelioma, head and neck cancer, non-small cell lung cancer, gastrointestinal stromal tumors, uterine leiomyoma and cutaneous T-cell lymphoma. A preferred group of NTR1 expressing tumor indications are pancreatic ductal adenocarcinoma, small cell lung cancer, prostate cancer, colorectal cancer, breast cancer, meningioma and Ewing's sarcoma.

Because of this selective expression of NTR1, NTR1 is regarded as a suitable target for therapeutic agents. Agonists and antagonists binding to NTR1 have been described in the prior art and WO2014/086499 describes an overview of such compounds.

WO2014/086499 also discloses a family of NTR1 antagonists suitable as diagnostic agents and/or pharmaceutical agents, particularly if conjugated to a diagnostic and/or therapeutic radionuclide.

Among the above-mentioned compounds, a first-in-human phase I study of ¹⁷⁷Lu-IPN01087 monotherapy (NCT03525392) is ongoing to assess preliminary safety and efficacy.

The combination of the invention intends to treat the tumours that overexpress neurotensin receptors, preferably pancreatic ductal adenocarcinoma and metastatic pancreatic ductal adenocarcinoma.

Pancreatic ductal adenocarcinoma (PDAC), which is the most common type of pancreatic cancer (95%), is a highly aggressive malignancy associated with a very poor clinical prognosis. With almost as many deaths (n=45,750) as new cases (n=56,770) in 2019, pancreatic cancer is currently the fourth leading cause of cancer death in the US, accounting for 7% of all cancer-related mortalities. The incidence and mortality rates of pancreatic cancer have been increasing for decades, and it is projected that pancreatic cancer will surpass liver, breast, prostate and colorectal cancers to become the second leading cause of cancer-related death by 2030. Currently, the Surveillance, Epidemiology, and End Results database reports the five-year survival rate for pancreatic cancer at 9.3%, which has improved only slightly in recent years, despite newer treatments. These statistics clearly highlight the need for new approaches to effectively treat this invariably fatal disease.

The poor prognosis of PDAC is mainly attributed to the lack of visible and specific symptoms and reliable biomarkers for early diagnosis, as well as an aggressive metastatic spread leading to poor response to treatments. According to the American Cancer Society (2019), more than half (52%) of patients with pancreatic cancer are diagnosed with metastatic disease. Furthermore, due to the broad heterogeneity of acquired genetic mutations and dense stromal environment, PDAC is highly resistant to current chemotherapy and radiotherapy regimens.

Since 1997, gemcitabine (CAS Registry No. 95058-81-4), a nucleoside metabolic inhibitor, has been the cornerstone of first-line treatment of patients with metastatic PDAC, after being shown to offer greater clinical benefit and a survival improvement over weekly 5-fluorouracil therapy. Gemcitabine is the active pharmaceutical ingredient in GEMZAR®.

Different gemcitabine-based combinations have since been intensely evaluated. However, only one combination showed anti-tumour activity and efficacy in metastatic PDAC first-line treatment: gemcitabine and nab-paclitaxel.

Nab-paclitaxel (CAS Registry No. 33069-62-4) is a nanoparticle albumin-bound formulation of paclitaxel (ABRAXANE®), which is a microtubule inhibitor. The regulatory approval in both the US and European Union of gemcitabine and nab-paclitaxel as a first-line therapy option for patients with metastatic PDAC was based on the findings of the MPACT phase III study, in which gemcitabine and nab-paclitaxel significantly improved overall survival (OS) (8.5 versus 6.7 months; p<0.001), progression-free survival (PFS) (5.5 versus 3.7 months; p<0.001), and overall response rate (ORR) (23% versus 7%; p<0.001), compared with gemcitabine alone.

Besides gemcitabine-based chemotherapy, FOLFIRINOX, a multiagent chemotherapy regimen composed of folinic acid (also known as leucovorin), fluorouracil (also known as 5-FU), irinotecan and oxaliplatin, has emerged as an alternative treatment strategy for patients with metastatic PDAC showing a survival advantage (median OS of 11.1 versus 6.8 months; median PFS of 6.4 versus 3.3 months; and ORR of 31.6% versus 9.4%; p<0.001 for all parameters) compared to standard single-agent gemcitabine in a randomized, multi-center, phase II/III study.

However, despite the recent improvements in the treatment of PDAC, initially with demonstration of the activity of the FOLFIRINOX regimen and subsequently the approval of gemcitabine in combination with nab-paclitaxel, prognosis remains poor with median OS times of less than one year. Due to the difficulty in early detection, most patients with pancreatic cancer have advanced disease that has spread, or metastasized, to other parts of the body, thus limiting the number of patients who are able to seek surgical resection as a treatment option. To date, neither personalized medicine nor immunotherapy, which are considered the two recent revolutions of cancer treatment, have delivered major positive results in the treatment of PDAC. Reasons for the failure of most targeted therapies might be the complex genetic mechanisms taking place in pancreatic tumour cells, which favor resistance to cytotoxic as well as targeted agents, and the intricate tumour microenvironment that seems to protect the tumour through a discrete vessel network and a hypoxic milieu. Likewise, immunotherapy approaches may find difficulty in entering the stroma and reaching the tumour cells.

Hence, other therapeutic options should be sought to improve the outcome of patients with PDAC. In addition, considering the aggressive nature of the disease and the high resistance of PDAC to standard of care chemotherapy, a combination therapy approach is biologically justified.

Besides PDAC, NTR1 is also highly expressed in colorectal cancer.

Colorectal cancer is the third most common type of cancer, making up about 10% of all cases. Survival is directly related to detection and the type of cancer involved, but overall is poor for symptomatic cancers, as they are typically quite advanced. Survival rates for early-stage detection are about five times that of late-stage cancers.

There exists a need for more and different therapies that may prove to be effective in treating the above-mentioned tumours that overexpress neurotensin receptor, such as, but not only, pancreatic ductal adenocarcinoma.

Novel combinations of therapeutic agents to treat neurotensin receptor over-expressing tumours are presented herein.

### SUMMARY

The present invention provides a combination comprising a neurotensin receptor binding compound and FOLFIRINOX for use for the treatment of a neurotensin receptor overexpressing tumour in a subject.

The invention also concerns a method for treating a neurotensin receptor over-expressing tumour in a subject, comprising administering to the subject an effective amount of a neurotensin receptor binding compound and FOLFIRINOX.

The invention also concerns the use of a neurotensin receptor binding compound for the manufacture of a medicament for treating a neurotensin receptor overexpressing tumour in a subject, in combination with FOLFIRINOX.

The invention also concerns a neurotensin receptor binding compound for use in treating a neurotensin receptor overexpressing tumour in a subject, wherein said neurotensin receptor binding compound is administered in combination with FOLFIRINOX.

### DETAILED DESCRIPTION

The embodiments of the present disclosure relate to a combination comprising a neurotensin receptor binding compound, folinic acid, fluorouracil, irinotecan and oxaliplatin, for use for the treatment of a neurotensin receptor overexpressing tumour in a subject.

According to the present invention, “FOLFIRINOX” refers to a chemotherapy regimen for treatment of advanced pancreatic cancer. It is made up of the following four drugs:
a) FOL—folinic acid (leucovorin), a vitamin B derivative that modulates/potentiates/reduces the side effects of fluorouracil;
b) F—fluorouracil (5-FU), a pyrimidine analog and antimetabolite which incorporates into the DNA molecule and stops DNA synthesis;
c) IRIN—irinotecan, a topoisomerase inhibitor, which prevents DNA from uncoiling and duplicating; and
d) OX—oxaliplatin, a platinum-based antineoplastic agent, which inhibits DNA repair and/or DNA synthesis.

### Neurotensin Receptor Binding Compound

According to a preferred embodiment, the neurotensin receptor binding compound is radiolabeled with a therapeutic radionuclide.

As used herein the term “radiolabeled” refers to a compound which is labelled with a radionuclide element, typically of metallic nature. Accordingly, a radiolabeled neurotensin receptor binding compound is a compound which comprises a radionuclide and which has specific binding affinity to neurotensin receptor. In some embodiments of the disclosure, said radiolabeled neurotensin receptor binding compound with specific binding affinity to at least NTR1 receptor.

More particularly, the neurotensin receptor binding compound comprises a neurotensin-targeting molecule linked to a chelating agent able to chelate the therapeutic radionuclide. Preferably, the chelating agent is covalently linked to the neurotensin-targeting molecule, either directly or via a linker.

As used herein, the term “neurotensin-targeting molecule” refers to a molecule with specific binding affinity to neurotensin receptor.

Preferably, the neurotensin receptor binding compound comprises a complex formed by a therapeutic radionuclide and a neurotensin-targeting molecule covalently linked to a chelating agent able to chelate the therapeutic radionuclide.

More preferably, the neurotensin receptor binding compound consists of a complex formed by a therapeutic radionuclide and a neurotensin-targeting molecule covalently linked to a chelating agent able to chelate the therapeutic radionuclide.

As used herein, the term “chelating agent” refers to an organic moiety comprising functional groups that are able to form non-covalent bonds with the radionuclide and, thereby, form stable radionuclide complex. Such chelating agents are either directly linked to the somatostatin receptor binding peptide or connected via a linker molecule, preferably it is directly linked. The linking bond(s) is (are) either covalent or non-covalent bond(s) between the cell receptor binding organic moiety (and the linker) and the chelating agent, preferably the bond(s) is (are) covalent.

The chelating agent can be selected from DOTA, NOTA, DTPA, DO3A, TETA, EDTA, NODAGA, NODASA, NOC, TRITA, CDTA, BAT, DFO, and HYNIC. The chelating agent is preferably DOTA.

Preferably, the neurotensin-targeting molecule is a neurotensin inhibitor, such as a neurotensin antagonist or a neurotensin agonist. More preferably, the neurotensin-targeting molecule is a NTR1 antagonist or a NTR1 agonist.

The therapeutic radionuclide is preferably selected from the beta-emitting radionuclides and the alpha-particle emitting radionuclides.

Beta-emitting radionuclides commonly used in cancer therapy comprise ¹⁷⁷Lu, ⁹⁰Y, ⁶⁷Cu, ¹³¹I, ¹⁸⁶Re, ¹⁸⁸Re, ²¹²Pb, and ²¹³Bi.

Alpha-particle emitting radionuclides commonly used in cancer therapy comprise ²¹¹At, ²¹²Pb, ²¹³Bi, ²²⁵Ac, and ²²⁷Th.

Preferably, the therapeutic radionuclide is selected from the group comprising ¹⁷⁷Lu, ⁹⁰Y, ⁶⁷Cu, ¹³¹I, ¹⁸⁶Re, ¹⁸⁸Re, ²¹¹At, ²¹²Pb, ²¹³Bi, ²²⁵Ac, and ²²⁷Th.

The therapeutic radionuclide is advantageously ¹⁷⁷Lu or ²²⁵Ac.

According to a preferred embodiment, the neurotensin receptor binding compound comprises a molecule of formula (i):

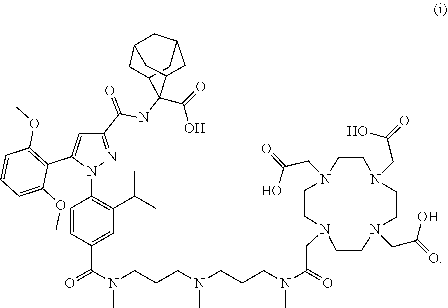 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]

or a complex thereof, preferably with a therapeutic radionuclide.

The compound of formula (i) is also known as IPN01087 or zalsenertant tetraxetan (CAS Registry No. 1613265-38-5).

The compound of formula (i) can be radiolabeled with all therapeutic radionuclides that can be chelated by DOTA chelator, such as ¹⁷⁷Lu, ⁹⁰Y, ⁶⁷Cu, and ²²⁵Ac. In some embodiments, the therapeutic radionuclide for chelating the compound of formula (i) is ¹⁷⁷Lu. In some embodiments, the therapeutic radionuclide for chelating the compound of formula (i) is ²²⁵Ac.

According to a preferred embodiment, the neurotensin receptor binding compound is a complex of formula (ii):

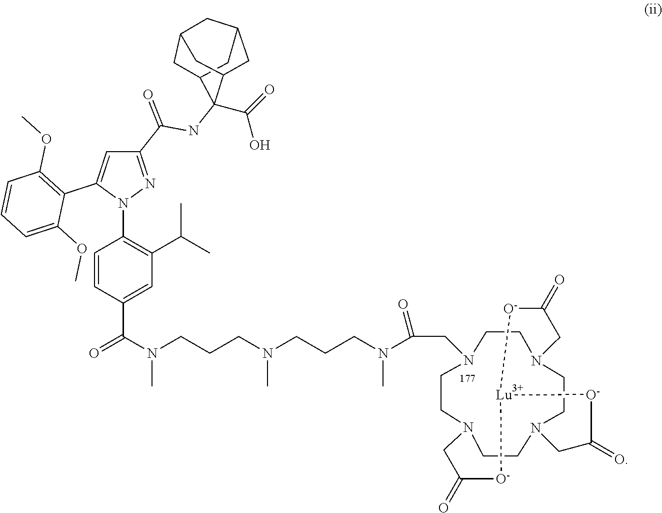

The compound of formula (i) is also known as ¹⁷⁷Lu-IPN01087, also referred to as ¹⁷⁷Lu-zalsenertant tetraxetan.

According to another embodiment, the neurotensin receptor binding compound is a compound of formula (I) as described in WO2014086499.

### Indication

The combination of the invention is useful for treating neurotensin receptor over-expressing tumours.

In particular, the neurotensin receptor overexpressing tumour can be selected in the group comprising pancreatic ductal adenocarcinoma (PDAC), small cell lung cancer, prostate cancer, colorectal cancer, breast cancer, meningioma, Ewing's sarcoma, pleural mesothelioma, head and neck cancer, non-small cell lung cancer, gastrointestinal stromal tumors, uterine leiomyoma and cutaneous T-cell lymphoma.

More particularly, the neurotensin receptor overexpressing tumour can be selected from pancreatic ductal adenocarcinoma and colorectal cancer. In some embodiments, the neurotensin receptor overexpressing tumour is pancreatic ductal adenocarcinoma. In some embodiments, the neurotensin receptor overexpressing tumour is colorectal cancer.

The combination of the invention is particularly useful for treating metastatic or unresectable pancreatic ductal adenocarcinoma.

According to an embodiment, the combination of the invention is particularly useful for treating neurotensin receptor overexpressing tumour resistant to treatment with FOLFIRINOX.

According to an embodiment, the method of treatment of the invention is for treating subjects with metastatic PDAC who have not previously received therapy for pancreatic cancer and who demonstrated uptake of ¹⁷⁷Lu-IPN01087 or ¹¹¹In-IPN01087 in the tumour lesions.

According to a preferred embodiment, the neurotensin receptor binding compound is a NTR1 binding compound.

### Dosing Regimen

In the combination according to the invention, the neurotensin receptor binding compound is preferably for use in simultaneous, separate, or sequential combination with folinic acid, fluorouracil, irinotecan and oxaliplatin, in the treatment of neurotensin receptor overexpressing tumour.

According to one embodiment, FOLFIRINOX is administered according to a dosing regimen consisting of:
Day 1: oxaliplatin 85 mg/m² IV over 120 minutes,
Day 1: irinotecan 180 mg/m² IV over 90 minutes,
Day 1: folinic acid 400 mg/m² IV over 120 minutes, followed by
Day 1: fluorouracil 400 mg/m² IV bolus, followed by
Day 1-2: fluorouracil 2400 mg/m² IV continuous infusion over 46 hours.

According to another embodiment, FOLFIRINOX is administered according to a dosage regimen consisting of:
Day 1: oxaliplatin 85 mg/m² IV over 120 minutes, followed by
Day 1: folinic acid 400 mg/m² IV over 120 minutes, followed (after 30 minutes) by
Day 1: irinotecan 135 mg/m² IV over 90 minutes, followed by
Day 1: fluorouracil 300 mg/m² IV bolus, followed by
Day 1-2: fluorouracil 2400 mg/m² IV continuous infusion over 46 hours.

According to a preferred embodiment, the neurotensin receptor binding compound is radiolabeled with a therapeutic radionuclide and is more preferably the compound of formula (ii).

According to this embodiment, the radiolabeled neurotensin receptor binding compound is administered by injection IV at a dose of about 2 to 7 GBq per injection. As used herein, the term “injection IV” refers to an intravenous (IV) injection.

Preferably, the radiolabeled neurotensin receptor binding compound is administered at a dose of about 2 GBq per injection, at a dose of about 2.5 GBq per injection, at a dose of about 3 GBq per injection, at a dose of about 3.5 GBq per injection, at a dose of about 4 GBq per injection, at a dose of about 4.5 GBq per injection, at a dose of about 5 GBq per injection, at a dose of about 5.5 GBq per injection, at a dose of about 6 GBq per injection, at a dose of about 6.5 GBq per injection, at a dose of about 7 GBq per injection, or at a dose of about 7.5 GBq per injection.

In some embodiments, the radiolabeled neurotensin receptor binding compound is administered as a unitary dosage of less than 40 MBq. In some embodiments, the radiolabeled neurotensin receptor binding compound is administered as a unitary dosage of 1-28 MBq (e.g., 3-25 MBq, 5-20 MBq, 5-15 MBq, or 10-15 MBq) to said subject. As used herein, “unitary dosage” typically refers to a single dose. To perform this invention, the radiolabeled neurotensin receptor binding compound can be administered as a unitary dosage for multiple times, i.e., administered multiple doses.

In some embodiments, the radiolabeled neurotensin receptor binding compound is the compound of formula (i) chelated with ²²⁵Ac. In some embodiments, the compound of formula (i) chelated with ²²⁵Ac is administered as a unitary dosage of about 5-15 MBq (e.g., about 5 MBq, about 6 MBq, about 7 MBq, about 8 MBq, about 9 MBq, about 10 MBq, about 11 MBq, about 12 MBq, about 13 MBq, about 14 MBq, or about 15 MBq).

### General Definitions

The use of the articles “a”, “an”, and “the” in both the description and claims are to be construed to cover both the singular and the plural, unless otherwise indicated herein or clearly contradicted by context. The terms “comprising”, “having”, “being of”, “including”, and “containing” are to be construed as open terms (i.e. meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about” has herein the meaning that the following value may vary for ±20%, preferably ±10%, more preferably ±5%, even more preferably ±2%, even more preferably ±1%.

Unless otherwise defined, “%” has herein the meaning of weight percent (wt %), also referred to as weight by weight percent (w/w %).

The phrase “treatment of” and “treating” includes the amelioration or cessation of a disease, disorder, or a symptom thereof. In particular, with reference to the treatment of a tumour, the term “treating” may refer to slowing or inhibiting the growth of the tumour, or the reducing the size of the tumour.

The terms “patient” and “subject” which are used interchangeably refer to a human being, including for example a subject that has cancer.

As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e. characterizing or constituting a disease state, or may be categorized as non-pathologic, i.e. a deviation from normal but not associated with a disease state. Unless specified otherwise, the term is meant to include all types of cancerous growths or oncogenic processes, metastatic tissues or malignantly transformed cells, tissues, or organs, irrespective of histopathologic type or stage of invasiveness.

“Combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

As used herein, “simultaneous” or “simultaneously” is used to mean that the two agents are administered concurrently.

As used herein, “sequential,” “sequentially,” “separate,” or “separately” is used to mean that the active agents are not administered concurrently, but one after the other. Thus, administration “sequential,” “sequentially,” “separate,” or “separately” may permit one agent to be administered within 5 minutes, 10 minutes or a matter of hours after the other provided the circulatory half-life of the first administered agent is such that they are both concurrently present in therapeutically effective amounts. The time delay between administrations of the components will vary depending on the exact nature of the components, the interaction there between, and their respective half-lives.

### BRIEF DESCRIPTION OF THE DRAWINGS

FIG. 1 represents the administration schedules from Groups 1 to 6.

FIG. 2 represents the tumor volume of mice bearing subcutaneous AsPC1 tumors from Groups 1, 2, 3 and 5 (Group 1=dots; Group 2=squares; Group 3=triangles; Group 5=diamonds), wherein Group 5 was treated with a combination according to the invention.

FIG. 3 represents the tumor volume of mice bearing subcutaneous AsPC1 tumors from Groups 1, 2, 4 and 6 (Group 1=dots; Group 2=squares; Group 4=triangles; and Group 6=diamonds), wherein Group 6 was treated with a combination according to the invention.

### EXAMPLES

### Materials & Methods

### Reference Substances

Leucovorin (folinic acid, Ca2+ complex) was dissolved with 0.9% NaCl solution to reach at 45 mg/mL final. 5-FU stock solution was diluted with 0.9% NaCl solution to reach 25 mg/mL concentration. Irinotecan was diluted in 0.9% NaCl solution to reach a final concentration of 3.75 mg/mL. Oxaliplatin stock solution was diluted with 5% glucose solution to reach 0.875 mg/mL final concentration.

Radiolabeling of IPN01087 with Lutetium-177

The radiolabeling procedure was performed using ammonium acetate 0.4 M containing 0.325 M gentisic acid pH 4.2 buffer and lutetium-177 (¹⁷⁷LuCl₃, ITG, specific activity >3,000 GBq/mg at calibration).

In a microtube, lutetium-177 (¹⁷⁷LuCl₃) was mixed with ammonium acetate 0.4 M containing 0.325 M gentisic acid pH 4.2 buffer (2.8× volume of lutetium-177 solution) and IPN01087 to reach a specific activity of 85 MBq/μg. The reaction mixture was incubated at +85° C. for 30 minutes using a heating system. At the end of the incubation period, the radiolabeling incorporation was assessed by reversed phase liquid chromatography and thin layer chromatography. The radiolabeling mixture was then diluted with ammonium acetate 0.4 M containing 0.325 M gentisic acid pH 4.2 buffer, 0.9% NaCl solution, and DTPA Ca(Na₃) to reach the desired radioactive doses in MBq and concentrations.

### Vehicles

The vehicle of Oxaliplatin is hereafter referred as vehicle #1. The vehicle of 5-FU and Folinic acid is hereafter referred as vehicle #2. The vehicle of ¹⁷⁷Lu-IPN01087 is hereafter referred as vehicle #3.

### Example 1: Efficacy Study on Animals Induced with AsPC1 Cells

### Cancer Cell Line

The AsPC1 cell line was isolated from a metastatic site (ascites) from a 62-year old female patient (Chen WH. et al., In Vitro. 1982 January; 18(1):24-34).

### Cell Culture Method

Tumor cells were grown as monolayer at 37° C. in a humidified atmosphere (5% C02, 95% air). The culture medium is RPMI 1640 containing glutamax supplemented with 10% heat inactivated fetal bovine serum, sodium pyruvate and hepes. For experimental use, tumor cells were detached from the culture flask using accutase and neutralized by addition of complete culture medium. The cells were counted and their viability was assessed by 0.25% trypan blue exclusion assay. Two frozen pellets of AsPC-1 tumor cells were prepared: one frozen cell pellet prepared during the in-vitro cell culture, and one frozen cell pellet prepared using the cell suspension used for tumor induction in mice.

### Animals

One hundred fifty (150) healthy female SWISS Nude mice (Crl:NU(Ico)-Foxn1 nu), 7 weeks old at reception, were obtained from Charles River.

### Tumor Induction

Tumors were induced by subcutaneous injection of ten million (107) AsPC-1 cells in 200 μL of RPMI 1640 medium containing matrigel (50:50, v:v, ref: 356237, BD Biosciences, France) into the right flank (in the axis of the heart) of 150 female animals. AsPC-1 tumor cell implantation was performed 24-72 hours after a whole body irradiation with a gamma-source (2 Gy (Nude mice), ⁶⁰Co, BioMep, France).

### Randomization

Animals were randomized by individual tumor volume when tumors reached a mean volume of 200-204 mm³. Fifty-six animals (56) out of 150 were randomized into 7 groups of 8 animals each using Vivo Manager® software (Biosystemes, France). Homogeneity between groups was tested by an analysis of variance (ANOVA). The day of randomization is designated as the day 0 (DO).

### Treatment

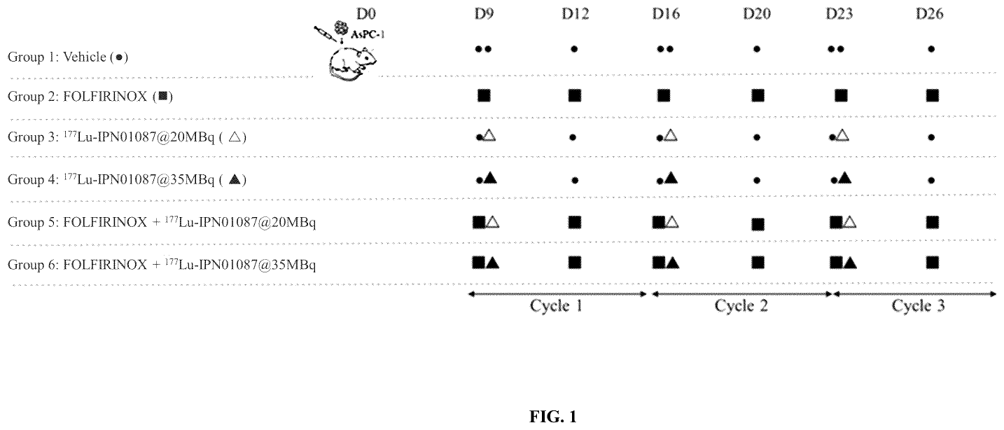

¹⁷⁷Lu-IPN01087 and the reference substances were administered by intravenous injection (IV) into the caudal vein via a catheter or by intraperitoneal (IP) injection. The recommended pH formulation for IV administration is pH 4.5-8.0 and for IP administration 7.3-7.4. The administration schedules were shown in FIG. 1.

The animal groups were treated as follows:
Group 1 (control group): animals received twice weekly IV injection of 0.5% glucose (vehicle #1) for a total of 6 injections combined with one weekly IP injection of 0.9% NaCl (vehicle #2) for a total of 3 injections, and one weekly IV injection of a radiolabeling buffer (vehicle #3) for a total of 3 injections.
Group 2 (“FOLFIRINOX” group): animals received twice weekly IV injection of Oxaliplatin at 1.75 mg/kg and Irinotecan at 7.5 mg/kg for a total of 6 injections, combined with one weekly IP injection of Folinic acid at 90 mg/kg and 5-FU at 50 mg/kg for a total of 3 injections each, and combined with one weekly IV injection of vehicle #3 for a total of 3 injections.
Group 3 (“¹⁷⁷Lu-IPN01087@20 MBq” group): animals received twice weekly IV injection of vehicle #1 for a total of 6 injections, combined with one weekly IP injection of vehicle #2 for a total of 3 injections, and one weekly IV injection of ¹⁷⁷Lu-IPN01087 at 20 MBq/mouse (0.443 nmol/mouse) for a total of 3 injections.
Group 4 (“¹⁷⁷Lu-IPN01087@35 MBq” group): animals received twice weekly IV injection of vehicle #1 for a total of 6 injections, combined with one weekly IP injection of vehicle #2 for a total of 3 injections, and one weekly IV injection of ¹⁷⁷Lu-IPN01087 at 35 MBq/mouse (0.776 nmol/mouse) for a total of 3 injections (treatment schedule: Q7Dx3).
Group 5 (“FOLFIRINOX+¹⁷⁷Lu-IPN01087@20 MBq” group): animals received twice weekly IV injection of Oxaliplatin at 1.75 mg/kg plus Irinotecan at 7.5 mg/kg for a total of 6 injections, combined with one weekly IP injection of Folinic acid at 90 mg/kg plus 5-FU at 50 mg/kg for a total of 3 injections each, and combined with one weekly IV injection of 177Lu-IPN01087 at 20 MBq/mouse (0.443 nmol/mouse) for a total of 3 injections.
Group 6 (“FOLFIRINOX+¹⁷⁷Lu-IPN01087@35 MBq” group): animals received twice weekly IV injection of Oxaliplatin at 1.75 mg/kg and Irinotecan at 7.5 mg/kg for a total of 6 injections, combined with one weekly IP injection of Folinic acid at 90 mg/kg and 5-FU at 50 mg/kg for a total of 3 injections, and combined with one weekly IV injection of 177Lu-IPN01087 at 35 MBq/mouse (0.776 nmol/mouse) for a total of 3 injections.

20 MBq in mice is equivalent to about 4.7 GBq in human (human equivalent dose).

35 MBq in mice is equivalent to about 7.6 GBq in human (human equivalent dose).

At the time of tumor relapse (or at termination), the tumor from 3 out of 8 mice from each group were collected. Tumors will be weighed, flash-frozen and then stored at −80° C.

The treatment efficacy was assessed in terms of the effects of the treatments on the tumor volumes of treated animals relative to control animals. The tumor volume was estimated by the formula:

$$Tumor Volume = \frac{{width}^{2} \times length}{2}$$

Tumors which were palpable and not measurable using calipers were assigned a volume of 4 mm³, indicating the technical limit measure. Tumor volume of 1000 mm³ were considered to be equal to 1 g. Individual, mean and median tumor volumes were measured.

Results are presented in FIGS. 1 to 3.

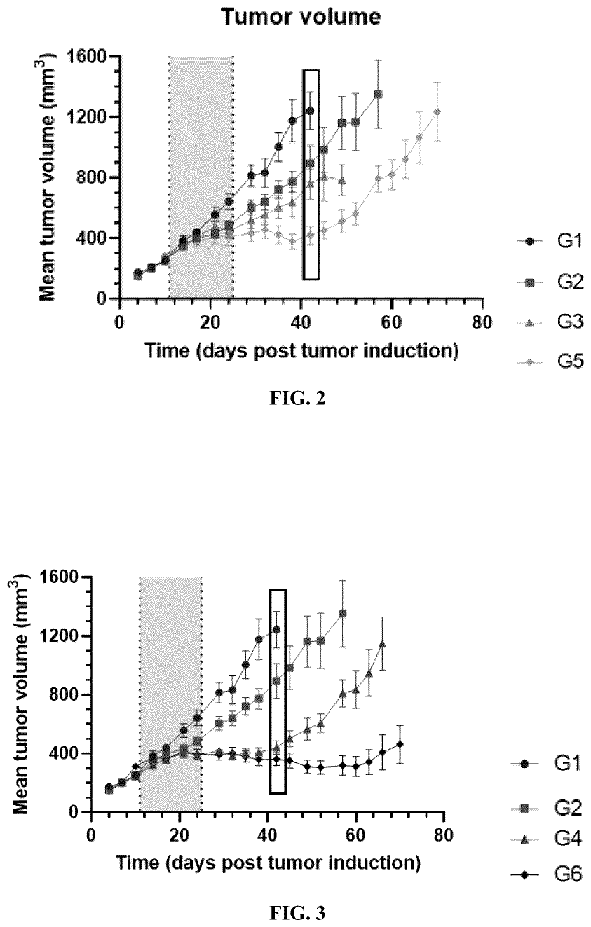

FIG. 2 shows AsPC-1 tumor growth in mice treated with vehicles (Group 1=dots), FOLFIRINOX (Group 2=squares), ¹⁷⁷Lu-IPN01087@20 MBq (Group 3=triangles), or a combination of FOLFIRINOX and ¹⁷⁷Lu-IPN01087 (Group 5=diamonds). Vertical dotted lines indicate the start and the end of the treatments. While FOLFIRINOX and ¹⁷⁷Lu-IPN01087 treatment alone produced an antitumor effect when compared to the vehicles, the combination of FOLFIRINOX and ¹⁷⁷Lu-IPN01087 significantly improved tumor growth control as compared to either single treatment.

Furthermore, the data shown in FIG. 3 demonstrate that a combination of FOLFIRINOX and ¹⁷⁷Lu-IPN01087 at a higher dose of 35 MBq (Group 6=diamonds) displayed even better tumor growth control as compared to FOLFIRINOX (Groupe 2=squares) and ¹⁷⁷Lu-IPN01087 (Group 4=triangles) alone.

Overall, the ¹⁷⁷Lu-IPN01087 radiotherapy in combination with the FOLFIRINOX chemotherapy regimen showed improved tumor growth control and prolonged tumor regression in AsPC-1 pancreatic cancer models as compared to the chemotherapy or radiotherapy regimens alone.

1. A method of treating a neurotensin receptor overexpressing tumor, comprising administering to a subject in need thereof a therapeutically effective amount of a combination comprising a neurotensin receptor binding compound, folinic acid, fluorouracil, irinotecan and oxaliplatin.

2. The method of claim 1, wherein the neurotensin receptor binding compound is radiolabeled with a therapeutic radionuclide.

3. The method of claim 2, wherein the therapeutic radionuclide is selected from the group consisting of ¹⁷⁷Lu, ⁹⁰Y, ⁶⁷Cu, ¹³¹I, ¹⁸⁶Re, ¹⁸⁸Re, ²¹¹At, ²¹²Pb, ²¹³Bi, ²²⁵Ac, and ²²⁷Th.

4. The method of claim 2, wherein the therapeutic radionuclide is ¹⁷⁷Lu or ²²⁵Ac.

5. The method of claim 1, wherein the neurotensin receptor binding compound comprises a molecule of formula (i): 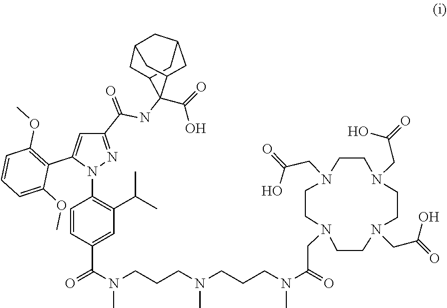 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]
or a complex thereof.

6. The method of claim 1, wherein the neurotensin receptor binding compound is radiolabeled with ²²⁵Ac.

7. The method of claim 1, wherein the neurotensin receptor binding compound is a complex of formula (ii):

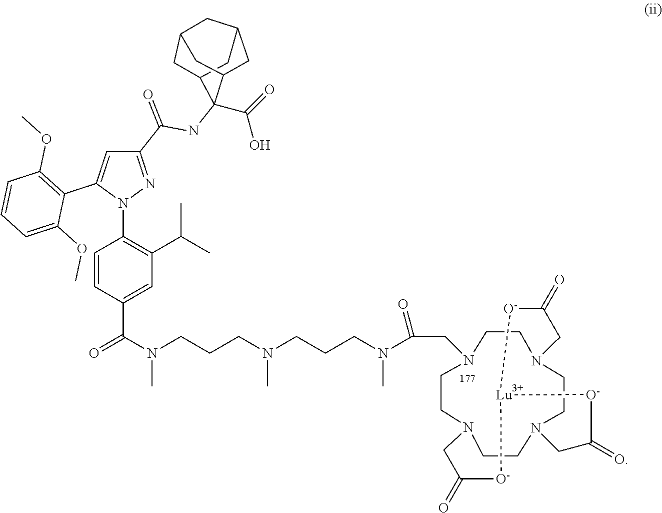

8. The method of claim 1, wherein the neurotensin receptor overexpressing tumour is pancreatic ductal adenocarcinoma or colorectal cancer.

9. The method of claim 1, wherein the neurotensin receptor binding compound is an NTR1 binding compound.

10. The method of claim 1, wherein the neurotensin receptor binding compound is administered in simultaneous, separate, or sequential combination with folinic acid, fluorouracil, irinotecan and oxaliplatin.

11. The method of claim 2, wherein the radiolabeled neurotensin receptor binding compound is administered by injection IV at a dose of about 2 to 7 GBq per injection.

12. The method of claim 2, wherein the radiolabeled neurotensin receptor binding compound is administered as a unitary dosage of less than 40 MBq.

13. The method of claim 2, wherein the therapeutic radionuclide is a beta-emitting radionuclide or an alpha-particle emitting radionuclide.

14. The method of claim 1, wherein the neurotensin receptor overexpressing tumor is selected from the group consisting of pancreatic ductal adenocarcinoma (PDAC), small cell lung cancer, prostate cancer, colorectal cancer, breast cancer, meningioma, Ewing's sarcoma, pleural mesothelioma, head and neck cancer, non-small cell lung cancer, gastrointestinal stromal tumors, uterine leiomyoma, and cutaneous T-cell lymphoma.

15. The method of claim 1, wherein the subject has metastatic PDAC and has not previously received a therapy for treating pancreatic cancer.
